(19) United States
(12) Patent Application Publication
(10) Pub. No.: US 2026/0272809 A1
(43) Pub. Date: Sep. 17, 2026

(54) CONTROLLING BACTERIAL CELL ADHESION WITH ALPHA-GULCAN COMPRISING ALPHA-1,6 GLYCOSIDIC LINKAGES

(71) Applicant: NUTRITION & BIOSCIENCES USA 4, INC., Wilmington, DE (US)

(72) Inventors: Jeffrey Wayne Munos, Palo Alto, CA (US); Bradley Kelemen; Cliff Lay; Helen S.M. Lu, Wilmington, DE (US); Arthur Ouwehand, Kotka (FI); Krista Salli, Kotka (FI)

(21) Appl. No.: 19/480,135

(22) PCT Filed: Apr. 29, 2024
(86) PCT No.: PCT/US2024/026773

§ 371 Date: Oct. 30, 2025

Related U.S. Application Data
(60) Provisional application No. 63/499,742, filed on May 3, 2023.
(60) Provisional application No. 63/572,553, filed on Apr. 1, 2024.

(51) Int. Cl. A61K 8/73; A61K 31/716; A61P 31/04; A61Q 11/00

(52) U.S. Cl. CPC A61K 8/73; A61K 31/716; A61P 31/04; A61Q 11/00; A61K 2800/92

(57) ABSTRACT

Disclosed herein are methods of inhibiting bacterial cells that comprise (a) providing a water-soluble alpha-glucan such as alpha-1,6-glucan, and (b) contacting the bacterial cells under aqueous conditions with the alpha-glucan and/or bringing the alpha-glucan into close proximity of the bacteria cells. Step (b) can lead to, for example, (i) inhibiting adhesion and/or colonization of the bacterial cells. These methods can optionally be applied to oral care applications, such as in preventing formation of dental caries. Further disclosed are oral care compositions and other compositions comprising water-soluble alpha-glucan such as alpha-1,6-glucan.

This application claims the benefit of U.S. Provisional Appl. Nos. 63/499,742 (filed May 3, 2023) and 63/572,553 (filed Apr. 1, 2024), which are each incorporated herein by reference in their entirety.

### FIELD

The present disclosure is in the field of polysaccharides and bacterial control. For example, the disclosure pertains to using one or more alpha-glucans with alpha-1,6 glycosidic linkages to control biofilm formation by bacteria.

### BACKGROUND

Driven by a desire to find new structural polysaccharides using enzymatic syntheses or genetic engineering of microorganisms, researchers have discovered oligosaccharides and polysaccharides that are biodegradable and that can be made economically from renewably-sourced feedstocks. Alpha-1,6-glucan (dextran), is an example of such advantaged material.

Dextran has been employed in various applications, for instance in personal care, household care, medical and pharmaceutical products, and food products. Various chemical derivatives of dextran have similarly been used. Despite this progress, still further uses for dextran are being pursued.

### SUMMARY

In one embodiment, the present disclosure concerns a method of inhibiting at least one bacterial cell. Such a method can comprise:
(a) providing at least one water-soluble alpha-glucan, wherein at least about 30% of the glycosidic linkages of the water-soluble alpha-glucan are alpha-1,6 linkages, and
(b) contacting at least one bacterial cell in aqueous conditions with the water-soluble alpha-glucan, wherein the contacting:
   (i) inhibits adhesion of the bacterial cell, and/or
   (ii) inhibits colonization by the bacterial cell.

In another embodiment, the present disclosure concerns an oral care composition/product comprising at least one water-soluble alpha-glucan, wherein at least about 30% of the glycosidic linkages of the water-soluble alpha-glucan are alpha-1,6 linkages, wherein the oral care composition/product has increased activity to:
(i) inhibit adhesion of a bacterial cell, and/or
(ii) inhibit colonization by a bacterial cell, wherein the increased activity is as compared to the activity of a reference/control oral care composition/product that does not comprise the water-soluble alpha-glucan.

### BRIEF DESCRIPTION OF THE DRAWINGS AND SEQUENCES

FIG. 1: Testing the effects of water-soluble alpha-glucans (Polymer 1, Polymer 2, or maltodextrin) on cell adhesion/biofilm formation by Streptococcus mutans in cultures containing sucrose. Polymer 1 (circles): dextran produced by GTF 6831. Polymer 2 (squares): dextran produced by combination of GTFs 6831 and 9905. Maltodextrin (triangles). Upside down triangles indicate results from cultures grown without sucrose. Refer to Example.

FIG. 2: Testing the effects of water-soluble alpha-glucans (Polymer 1, Polymer 3, or maltodextrin) on cell adhesion/biofilm formation by S. mutans in cultures containing sucrose. Polymer 1 (squares): dextran produced by GTF 6831. Polymer 3 (triangles): dextran produced by GTF 0768. Maltodextrin (diamonds). Upside down triangles indicate results from cultures grown without sucrose. Refer to Example.

### DETAILED DESCRIPTION

The disclosures of all cited patent and non-patent literature are incorporated herein by reference in their entirety.

Unless otherwise disclosed, the terms “a”, “an” and “the” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like.

A “glucan” herein is a type of polysaccharide that is a polymer of glucose (polyglucose). A glucan can be comprised of, for example, about, or at least about, 90%, 91%, 92%, 93%, 94%, 95%, 96%, 97%, 98%, 99%, or 100% by weight glucose monomeric units. Examples of glucans herein are alpha-glucan and beta-glucan.

The terms “alpha-glucan”, “alpha-glucan polymer” and the like are used interchangeably herein. An alpha-glucan is a polymer comprising glucose monomeric units linked together by alpha-glycosidic linkages. In typical embodiments, an alpha-glucan herein comprises at least about 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. Examples of alpha-glucan polymers herein include graft copolymers as presently disclosed, as well as alpha-1,3-glucan and alpha-1,6-glucan.

The term “saccharide” and other like terms herein refer to monosaccharides and/or disaccharides/oligosaccharides, unless otherwise noted. A “disaccharide” herein refers to a carbohydrate having two monosaccharides joined by a glycosidic linkage. An “oligosaccharide” herein can refer to a carbohydrate having 3 to 15 monosaccharides, for example, joined by glycosidic linkages. An oligosaccharide can also be referred to as an “oligomer”. Monosaccharides (e.g., glucose and/or fructose) comprised within disaccharides/oligosaccharides can be referred to as “monomeric units”, “monosaccharide units”, or other like terms.

The terms “dextran”, “dextran polymer”, “dextran molecule”, “alpha-1,6-glucan”, “alpha-glucan with alpha-1,6 linkages” and the like herein refer to water-soluble alpha-glucan comprising glucose monomeric units linked together by glycosidic linkages, wherein at least about 40% of the glycosidic linkages are alpha-1,6. Alpha-1,6-glucan in some aspects comprises about, or at least about, 90%, 95%, or 100% alpha-1,6 glycosidic linkages. Other linkages that can be present in alpha-1,6-glucan include alpha-1,2, alpha-1,3, and/or alpha-1,4 linkages. A “substantially linear” (“mostly linear”, and like terms) dextran herein has 5% or less branches, while a “linear” dextran has no branches. Dextran branches can be short, being one (pendant) to three glucose monomers in length, for example. Yet, in some aspects, dextran can be “dendritic”, which is a branched structure emanating from a core in which there are chains (containing mostly or all alpha-1,6-linkages) that iteratively branch from each other (e.g., a chain can be a branch from another chain, which in turn is a branch from another chain, and so on). Yet, in still some aspects, dextran is not dendritic, but has a branch-on-branch structure that does not emanate from a core. Enzymes capable of synthesizing dextran from sucrose may be described as “dextransucrases” (EC 2.4.1.5).

An “alpha-1,2 branch” (and like terms) as referred to herein typically comprises a glucose that is alpha-1,2-linked to a dextran backbone; thus, an alpha-1,2 branch herein can also be referred to as an alpha-1,2,6 linkage. An alpha-1,2 branch herein typically has one glucose group (can optionally be referred to as a pendant glucose). However, in some aspects, instead of there only being a pendant glucose, an alpha-1,2 branch can further comprise one or more glucose units linked in a chain extending from the alpha-1,2-linked glucose (i.e., a “side chain” or “side arm” of the dextran). Such a chain (or one glucose unit) typically is linked entirely through alpha-1,6 linkage(s) from (or “off of”) the alpha-1,2-linked glucose.

An “alpha-1,3 branch” (and like terms) as referred to herein typically comprises a glucose that is alpha-1,3-linked to a dextran backbone; thus, an alpha-1,3 branch herein can also be referred to as an alpha-1,3,6 linkage. An alpha-1,3 branch herein typically has one glucose group (can optionally be referred to as a pendant glucose). However, in some aspects, instead of there only being a pendant glucose, an alpha-1,3 branch can further comprise one or more glucose units linked in a chain extending from the alpha-1,3-linked glucose (i.e., a “side chain” or “side arm” of the dextran). Such a chain (or one glucose unit) typically is linked entirely through alpha-1,6 linkage(s) from (or “off of”) the alpha-1,3-linked glucose.

The percent branching in an alpha-glucan herein refers to that percentage of all the linkages in the alpha-glucan that represent branch points. For example, the percent of alpha-1,2 branching in an alpha-glucan herein refers to that percentage of all the linkages in the glucan that represent alpha-1,2 branch points. Except as otherwise noted, linkage percentages disclosed herein are based on the total linkages of an alpha-glucan, or the portion of an alpha-glucan for which a disclosure specifically regards.

The terms “linkage”, “glycosidic linkage”, “glycosidic bond” and the like refer to the covalent bonds connecting the sugar monomers within a saccharide compound (oligosaccharides and/or polysaccharides). Examples of glycosidic linkages include 1,6-alpha-D-glycosidic linkages (herein also referred to as “alpha-1,6” linkages), 1,3-alpha-D-glycosidic linkages (herein also referred to as “alpha-1,3” linkages), 1,4-alpha-D-glycosidic linkages (herein also referred to as “alpha-1,4” linkages), and 1,2-alpha-D-glycosidic linkages (herein also referred to as “alpha-1,2” linkages).

The glycosidic linkage profile of an alpha-glucan herein can be determined using any method known in the art. For example, a linkage profile can be determined using methods using nuclear magnetic resonance (NMR) spectroscopy (e.g., ¹³C NMR or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, FL, 2005), which is incorporated herein by reference.

The “molecular weight” of an alpha-glucan herein can be represented as weight-average molecular weight (Mw) or number-average molecular weight (Mn), the units of which are in Daltons (Da) or grams/mole. In some aspects, molecular weight can be represented as DPw (weight average degree of polymerization) or DPn (number average degree of polymerization). DPw and DPn are calculated from the corresponding Mw or Mn, respectively, by dividing by the molar mass of one monomer unit M₁. In the case of glucan polymer, M₁=162.14. In some aspects, molecular weight can sometimes be provided as “DP” (degree of polymerization), which simply refers to the number of glucoses comprised within the alpha-glucan on an individual molecule basis. Various means are known in the art for calculating these various molecular weight measurements such as with high-pressure liquid chromatography (HPLC), size exclusion chromatography (SEC), or gel permeation chromatography (GPC).

As used herein, Mw can be calculated as Mw=ΣNiMi²/ΣNiMi, where Mi is the molecular weight of an individual chain i and Ni is the number of chains of that molecular weight. Besides SEC, the Mw of a polymer can be determined by other techniques such as static light scattering, mass spectrometry, MALDI-TOF (matrix-assisted laser desorption/ionization time-of-flight), small angle X-ray or neutron scattering, or ultracentrifugation. As used herein, Mn can be calculated as Mn=YNiMi/YNi where Mi is the molecular weight of a chain i and Ni is the number of chains of that molecular weight. Besides SEC, the Mn of a polymer can be determined by various colligative property methods such as vapor pressure osmometry, end-group determination by spectroscopic methods such as proton NMR, proton FTIR, or UV-Vis. As used herein, DPw and DPn can be calculated from Mw and Mn, respectively, by dividing them by molar mass of the one monomer unit M₁. In the case of glucan polymer herein, M₁=162.

The term “sucrose” herein refers to a non-reducing disaccharide composed of an alpha-D-glucose molecule and a beta-D-fructose molecule linked by an alpha-1,2-glycosidic bond. Sucrose is known commonly as table sugar. Sucrose can alternatively be referred to as “alpha-D-glucopyranosyl-(1→2)-beta-D-fructofuranoside”. “Alpha-D-glucopyranosyl” and “glucosyl” are used interchangeably herein.

The terms “glucosyltransferase”, “glucosyltransferase enzyme”, “GTF”, “glucansucrase” and the like are used interchangeably herein. The activity of a glucosyltransferase herein catalyzes the reaction of the substrate sucrose to make the products alpha-glucan and fructose. Other products (by-products) of a GTF reaction can include glucose, various soluble gluco-oligosaccharides, and leucrose. Wild type forms of glucosyltransferase enzymes generally contain (in the N-terminal to C-terminal direction) a signal peptide (which is typically removed by cleavage processes), a variable domain, a catalytic domain, and a glucan-binding domain. A glucosyltransferase herein is classified under the glycoside hydrolase family 70 (GH70) according to the CAZy (Carbohydrate-Active EnZymes) database (Cantarel et al., Nucleic Acids Res. 37:D233-238, 2009). The term “dextransucrase” (and like terms) can optionally be used to characterize a glucosyltransferase enzyme that produces dextran.

The term “glucosyltransferase catalytic domain” herein refers to the domain of a glucosyltransferase enzyme that provides alpha-glucan-synthesizing activity to a glucosyltransferase enzyme. A glucosyltransferase catalytic domain typically does not require the presence of any other domains to have this activity.

The terms “enzymatic reaction”, “glucosyltransferase reaction”, “glucan synthesis reaction”, “reaction composition”, “reaction formulation” and the like are used interchangeably herein and generally refer to a reaction that initially comprises water, sucrose, at least one active glucosyltransferase enzyme, and optionally other components. Components that can be further present in a glucosyltransferase reaction typically after it has commenced include fructose, glucose, leucrose, soluble gluco-oligosaccharides (e.g., DP2-DP7) (such may be considered as products or by-products, depending on the glucosyltransferase used), and/or insoluble alpha-glucan product(s) of DP8 or higher. The term “under suitable reaction conditions” as used herein refers to reaction conditions that support conversion of sucrose to alpha-glucan product(s) via glucosyltransferase enzyme activity. It is during such a reaction that glucosyl groups originally derived from the input sucrose are enzymatically transferred and used in alpha-glucan polymer synthesis, glucosyl groups as involved in this process can thus optionally be referred to as the glucosyl component or moiety (or like terms) of a glucosyltransferase reaction.

The term “in situ” as used herein characterizes a glucosyltransferase reaction(s) made to occur inside (i) an oral cavity or (ii) an oral care product (or oral care product ingredient), whereby one or more isolated dextransucrases are provided in the oral cavity or oral care product/ingredient in which the enzyme(s) uses sucrose and water to produce alpha-1,6-glucan. In situ production of alpha-1,6-glucan in this manner typically substitutes for adding isolated alpha-1,6-glucan to an oral cavity and/or an oral care product/ingredient, though such addition can be performed if desired (e.g., to supplement the alpha-1,6-glucan produced in situ).

The terms “aqueous liquid”, “aqueous fluid”, “aqueous conditions”, “aqueous setting”, “aqueous system” and the like as used herein can refer to water or an aqueous solution. An “aqueous solution” herein can comprise one or more dissolved salts, where the maximal total salt concentration can be about 3.5 wt % in some embodiments. Although aqueous liquids herein typically comprise water as the only solvent in the liquid, an aqueous liquid can optionally comprise one or more other solvents (e.g., polar organic solvent) that are miscible in water. Thus, an aqueous solution can comprise a solvent having at least about 10 wt % water.

An “aqueous composition” herein has a liquid component that comprises about, or at least about, 10, 20, 30, 40, 50, 60, 70, 80, 90, 95, 99, or 100 wt % water, for example. Examples of aqueous compositions include mixtures, solutions, dispersions (e.g., suspensions, colloidal dispersions) and emulsions, for example. In some embodiments, the pH of an aqueous composition is between ~4 and ~9 (e.g., between ~6 and ~8).

An alpha-glucan herein that is “soluble”, “aqueous-soluble”, or “water-soluble” (and like terms) dissolves (or appreciably dissolves) in water or other aqueous conditions, optionally where the aqueous conditions are further characterized to have a pH of 4-9 (e.g., pH 6-8) and/or temperature of about 1 to 130° C. (e.g., 20-25° C., 35-40° C.). In contrast, an alpha-glucan that is “insoluble”, “aqueous-insoluble”, “water-insoluble” and the like does not dissolve under these conditions.

The term “viscosity” as used herein refers to the measure of the extent to which a fluid (aqueous or non-aqueous) resists a force tending to cause it to flow. Various units of viscosity that can be used herein include centipoise (cP, cps) and Pascal-second (Pa·s), for example. A centipoise is one one-hundredth of a poise; one poise is equal to 0.100 kg·m⁻¹·s⁻¹. The terms “viscosity modifier”, “viscosity-modifying agent”, “rheology modifier”, and the like herein refer to anything that can alter/modify the viscosity of a fluid or aqueous composition.

The terms “biofilm”, “surface-attached community of microbes” and the like herein refer to a collective/assemblage/population of one or more types of microbial cells (e.g., bacteria) associated with a surface. The cells in a biofilm typically are comprised within a matrix/scaffold of protein and extracellular polymeric substance(s) (EPS) such as polysaccharide material. A biofilm matrix can also comprise, in some aspects, noncellular materials such as mineral crystals, corrosion particles, clay or silt particles, and/or other components. Biofilms typically adhere to surfaces submerged in, or subjected to, aqueous conditions. Biofilms have been described, for example, by Davey and O'Toole (2000, Microbiol. Mol. Biol. Rev. 64:847-867), Donlan (2002, Emerg. Infect. Dis. 8:881-890), Satpathy et al. (2016, Biocatal. Agric. Biotechnol. 7:56-66), Beech and Cheung (1995, Int. Biodeter. Biodegr. 35:59-72), US2018/0187175, US2020/0308592, or US20220306968 (WO2020/247582), which are all incorporated herein by reference. “Plaque” (“dental plaque”) herein is a type of biofilm.

The term “cariogenic” (and similar terms) as used herein characterizes bacteria that can produce or promote the development of tooth decay (e.g., dental caries/cavities). Cariogenic bacteria typically can cause tooth decay by a process that involves the formation of plaque on dental (teeth) surfaces.

The term “planktonic cells” and like terms herein refer to microbial cells (e.g., bacteria) floating as single cells in a liquid medium. As opposed to biofilm cells, planktonic cells typically live freely and are not associated with other cells in a matrix. A single type of bacteria can exist either in a planktonic or biofilm state, depending on environmental cues and/or gene expression, for example.

An “oral care composition” or “oral care product” (and like terms) herein is any composition suitable for treating a soft or hard surface in an oral cavity such as dental (teeth) and/or gum surfaces.

The terms “sequence identity”, “identity” and the like as used herein with respect to a polypeptide amino acid sequence (e.g., that of a glucosyltransferase) are as defined and determined in U.S. Patent Appl. Publ. No. 2017/0002336, for example, which is incorporated herein by reference.

Various polypeptide amino acid sequences are disclosed herein as features of certain embodiments. Variants of these sequences that are at least about 70-85%, 85-90%, or 90%-95% identical to the sequences disclosed herein can be used or referenced. Alternatively, a variant amino acid sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identity with a sequence disclosed herein. The variant amino acid sequence has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, or 99% of the function/activity of the disclosed sequence.

A composition herein that is “dry” or “dried” typically has less than 5, 4, 3, 2, 1, 0.5, or 0.1 wt % water comprised therein.

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “weight/volume percent”, “w/v %” and the like are used interchangeably herein. Weight/volume percent can be calculated as: ((mass [g] of material)/(total volume [mL] of the material plus the liquid in which the material is placed))×100%. The material can be insoluble in the liquid (i.e., be a solid phase in a liquid phase, such as with a dispersion), or soluble in the liquid (i.e., be a solute dissolved in the liquid).

Compositions and processes/methods herein are not naturally occurring, and thus can optionally be characterized as being “isolated”. It is believed that the embodiments disclosed herein are synthetic/man-made (could not have been made except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein.

Some embodiments of the present disclosure concern a method of inhibiting (controlling, preventing, blocking) at least one bacterial cell. Such a method can comprise at least:
(a) providing a water-soluble alpha-glucan, wherein at least about 30% of the glycosidic linkages of the water-soluble alpha-glucan are alpha-1,6 linkages (i.e., the alpha-glucan is alpha-1,6-glucan/dextran herein), and
(b) contacting at least one bacterial cell in aqueous conditions with the water-soluble alpha-glucan and/or bringing the alpha-glucan into close proximity of the bacterial cell, wherein step (b):
(i) inhibits adhesion of the bacterial cell (e.g., adhesion to a surface and/or to another bacterial cell),
(ii) inhibits colonization by the bacterial cell (e.g., inhibits biofilm formation by the bacterial cell), and/or
(iii) inhibits formation of extracellular matrix (ECM) by the bacterial cell.
   Such a method/process can optionally be characterized herein as a bacterial cell anti-adhesion method (or like terms), or, if practiced in an oral cavity, as an anti-oral plaque method, plaque control method, or other like terms.

In some aspects, a water-soluble alpha-glucan comprises about, or at least about, 30%, 40%, 50%, 60%, 70%, 80%, 85%, 90%, 91%, 92%, 93%, 94%, 95%, 96%, 97%, 98%, 99%, 99.5%, or 100% alpha-1,6 glycosidic linkages (i.e., the alpha-glucan is alpha-1,6-glucan/dextran). In some aspects, a dextran is a substantially linear dextran, that comprises 5%, 4%, 3%, 2%, 1%, 0.5%, or less glycosidic branches (e.g., a linear dextran can have about 100% alpha-1,6 linkages). If present, glycosidic branches from a dextran are typically short, being one (pendant), two, or three glucose monomers in length. In some aspects, a dextran can comprise about, or less than about, 50%, 40%, 30%, 20%, 15%, 10%, 9%, 8%, 7%, 6%, 5%, 4%, 3%, 2%, 1%, 0.5%, or 0% alpha-1,4, alpha-1,3 and/or alpha-1,2 glycosidic linkages. Typically, such linkages exist entirely, or almost entirely, as branch points from dextran.

Dextran herein can have alpha-1,2, alpha-1,3, and/or alpha-1,4 branches, for example. A dextran can only have alpha-1,2 branches in some aspects. A dextran can only have alpha-1,3 branches in some aspects. In some aspects, about, at least about, or less than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 2-25%, 2-20%, 2-15%, 2-10%, 3-25%, 3-20%, 3-15%, 3-10%, 5-30%, 5-25%, 5-20%, 5-15%, 5-10%, 7-13%, 8-12%, 9-11%, 10-30%, 10-25%, 10-20%, 10-15%, 10-22%, 15-30%, 15-25%, 15-20%, 20-30%, 20-25%, 12-20%, 12-18%, 14-20%, 14-18%, 15-18%, or 15-17% of all the glycosidic linkages of a branched dextran are alpha-1,2, alpha-1,3, and/or alpha-1,4 glycosidic branch linkages. Such branches typically are mostly (>90%, >95%, or >99%), or all (100%), a single glucose unit in length. In some aspects, dextran with alpha-1,2-branching can be produced enzymatically according to the procedures in U.S. Patent Appl. Publ. Nos. 2017/0218093 or 2018/0282385 (both incorporated herein by reference) where, for example, an alpha-1,2-branching enzyme such as GTFJ18T1 or GTF9905 can be added during or after production of the dextran. In some aspects, any other enzyme known to produce alpha-1,2-branching can be used. Dextran with alpha-1,3-branching can be prepared, for example, as disclosed in Vuillemin et al. (2016, J. Biol Chem. 291:7687-7702) or U.S. Patent Appl. Publ. No. 2022/0267745, which is incorporated herein by reference.

Dextran herein can have a DPw, DPn, or DP of about, at least about, or less than about, 6, 10, 25, 50, 100, 250, 500, 1000, 1500, 2000, 2500, 3000, 10-100, 10-250, 10-500, 10-1000, 10-1500, 10-2000, 10-2500, 10-3000, 25-100, 25-250, 25-500, 25-1000, 25-1500, 25-2000, 25-2500, 25-3000, 50-75, 50-100, 50-250, 50-500, 50-1000, 50-1500, 50-2000, 50-2500, 50-3000, 100-100, 100-250, 100-400, 100-500, 100-1000, 100-1500, 100-2000, 100-2500, 100-3000, 200-300, 225-275, 250-500, 250-1000, 250-1500, 250-2000, 250-2500, 250-3000, 500-1000, 500-1500, 500-2000, 500-2500, 500-3000, 750-1000, 750-1500, 750-2000, 750-2500, 750-3000, 1000-1250, 1000-1500, 1000-2000, 1000-2500, 1000-3000, or 1200-1300, for example. The molecular weight of dextran in some aspects can be about, at least about, or less than about, 1, 5, 7.5, 10, 12.5, 15, 20, 30, 40, 50, 60, 70, 80, 90, 100, 125, 150, 200, 225, 250, 275, 300, 400, 500, 1-500, 1-400, 1-300, 1-200, 1-100, 1-50, 7.5-12.5, 10-500, 10-400, 10-300, 10-200, 10-100, 10-50, 25-500, 25-400, 25-300, 25-200, 25-100, 25-50, 50-500, 50-400, 50-300, 50-200, 50-100, 100-500, 100-400, 100-300, 100-200, 150-500, 150-400, 150-300, 150-250, 150-225, 150-200, 175-225, 200-500, 200-400, 200-300, 200-250, or 200-225 kDa, for example. The molecular weight of dextran can be calculated, if desired, based on any of the foregoing dextran DPw, DPn, or DP values. Any of the forgoing DPw, DPn, DP, or Dalton values/ranges can characterize a dextran herein before, or after, it has optionally been branched (e.g., alpha-1,2 and/or alpha-1,3), for instance.

Dextran herein can be as disclosed (e.g., molecular weight, linkage/branching profile, production method), for example, in any of U.S. Patent Appl. Publ. Nos. 2016/0122445, 2017/0218093, 2018/0282385, 2020/0165360, or 2019/0185893, which are each incorporated herein by reference. In some aspects, a dextran can be one produced in a suitable reaction comprising glucosyltransferase (GTF) 8117, 6831, or 5604 (these three GTF enzymes are SEQ ID NOs:30, 32 and 33, respectively, of US2018/0282385), or a GTF comprising an amino acid sequence that is at least 90%, 91%, 92%, 93%, 94%, 95%, 96%, 97%, 98%, 99%, or 99.5% identical to the amino acid sequence of GTF 8117, 6831, or 5604.

In some aspects, dextran can have been produced in an isolated reaction comprising at least: (i) water, (ii) sucrose, and (iii) at least one isolated glucosyltransferase enzyme that synthesizes alpha-1,6-glucan (i.e., a dextransucrase herein). Such an isolated reaction can be conducted in an inert vessel/container (e.g., made of steel or stainless steel, glass, synthetic material such as plastic) and/or under cell-free conditions or conditions in which a cell is not used to produce the dextran (e.g., the dextran is not a product of a bacterial culture or bacterial fermentation process), for example. One, two, three, or more different dextransucrase enzymes (e.g., enzymes derivable from one or more different bacterial species, enzymes with different amino acid sequences) can be used in an isolated reaction, for example. Dextran produced in this manner can have any features disclosed herein, such as linkage profile, branching profile, and/or molecular weight. A dextransucrase (or any other enzyme as presently disclosed) for use in a method herein is typically in purified/isolated form. A purified/isolated enzyme can be essentially free from insoluble and/or soluble components of an organism/cell used to produce the enzyme, and/or any medium that was used for cellular fermentation of the enzyme. In some aspects, a purified/isolated enzyme denotes an enzyme preparation that contains less than 10%, 8%, 6%, 5%, 4%, 3%, 2%, 1%, 0.5%, 0.1%, 0.05%, or 0.01% by weight of other material (e.g., cellular material such as polypeptide material, genetic material, and/or lipid material) with which the enzyme was natively or recombinantly/heterologously associated. Typically, an isolated dextransucrase and/or any other enzyme herein as used in an isolated reaction is not comprised in or otherwise associated with (e.g., expressed by) a microbial (e.g., bacterial, yeast, fungal, algal) cell. However, in some aspects, a dextransucrase and/or any other enzyme herein is comprised in or otherwise associated with a microbial cell such as one that heterologously expresses the enzyme(s) (i.e., recombinant cells), which microbial cell is used as a means for providing dextran in a bacterial cell anti-adhesion method herein (e.g., for producing dextran as presently disclosed in a body cavity such as on oral cavity in an in situ manner).

A dextran in some aspects can have: (i) a 9-15, 10-14, or ~12 kDa backbone (i.e., before branching) and about 35-45%, 38-42%, or ~40% alpha-1,2 branching; (ii) a 11-17, 12-16, or ~14 kDa backbone (i.e., before branching) and about 15-25%, 18-22%, or ~20% alpha-1,2 branching; (iii) a 35-45, 37-43, or ~40 kDa backbone (i.e., before branching) and about 15-25%, 18-22%, or ~20% alpha-1,2 branching; (iv) a 185-215, 190-210, or ~200 kDa backbone (i.e., before branching) and about 3-7%, 4-6%, or ~5% alpha-1,2 branching; (v) a 185-215, 190-210, or ~200 kDa backbone (i.e., before branching) and about 15-25%, 18-22%, or ~20% alpha-1,2 branching; or (vi) a 7-13, 8-12, or ~10 kDa backbone (i.e., before branching) and about 10-20%, 12-18%, or ~15% alpha-1,3 branching.

In some aspects, dextran comprises one or more alpha-1,2 branches (e.g., percent thereof based on any alpha-1,2 linkage percentage disclosed herein), wherein at least one of the branches comprises a side chain (meaning a side chain to the dextran backbone from which the at least one branch originates; i.e., the at least one branch is the side chain), wherein at least about 50%, 60%, 70%, 80%, 90%, 95%, 99%, or 100% of the glycosidic linkages of the side chain (not counting the alpha-1,2 linkage to the dextran backbone) are alpha-1,6 linkages and/or each side chain itself has a molecular weight (and/or degree of polymerization) as disclosed herein for a dextran. In this sense, such a dextran can optionally be characterized as a grafted polymer or grafted dextran polymer, having a dextran backbone onto which one or more dextran side chains are linked via alpha-1,2-linkage. In some aspects, a dextran with this grafted polymer structure can further have one or more iterations of alpha-1,2-linked dextran side chains, wherein the dextran can optionally be characterized to contain a branch-on-branch structure (such branching either does, or does not, emanate from a core). The total glycosidic linkage profile and/or molecular weight (and/or degree of polymerization) of such a grafted dextran polymer can be as disclosed herein for dextran, for example.

Typically, a grafted dextran polymer as described above can be produced in an isolated dextransucrase reaction (above) that further comprises at least one alpha-1,2-branching enzyme such as disclosed herein. For example, an isolated reaction can comprise GTF 6831 (or GTF 8117 or GTF 5604) as the dextransucrase and GTF 9905 as the alpha-1,2-branching enzyme. In some other aspects, a grafted dextran polymer can have all the features as described above, except for having alpha-1,3 (or both alpha-1,3 and alpha-1,2) linkage branch points instead of alpha-1,2 linkage branch points.

Dextran as provided herein typically does not have any chemical derivatization (e.g., etherification, esterification, phosphorylation, sulfation, oxidation, carbamation) (e.g., no substitution of hydrogens of dextran hydroxyl groups with a non-sugar chemical group). In some aspects, dextran as provided herein is not bound or otherwise linked to a carrier/nanoparticle (i.e., the dextran is free).

A bacterial cell anti-adhesion method of the present disclosure comprises contacting at least one bacterial cell in aqueous conditions (an aqueous setting) with the water-soluble alpha-glucan and/or bringing the alpha-glucan into close proximity of the bacterial cell, or otherwise exposing the bacterial cell to the alpha-glucan in such a manner as to inhibit adhesion and/or colonization of the bacterial cell. By “close proximity”, it is meant the distance (and all lower distances) between a bacterial cell and the water-soluble alpha-glucan (dextran) for which the dextran inhibits adhesion and/or colonization of the bacterial cell. Such a distance can be about, or less than about, 0.001, 0.005, 0.01, 0.05, 0.1, 0.5, 1, 2, 0.001-2, 0.001-1, 0.001-0.5, 0.001-0.1, or 0.001-0.05 m (micron), for example.

The aqueous conditions in which a bacterial cell is exposed to dextran herein typically comprise at least sucrose. In some aspects, the concentration of sucrose in the aqueous conditions herein can be about, or at least about, 0.001, 0.005, 0.01, 0.05, 0.1, 0.5, 1, 2.5, 5, 10, 0.01-2.5, 0.01-1, 0.01-0.5, 0.1-2.5, 0.1-1, 0.1-0.5, or 1-2.5 wt %.

Aqueous conditions in some aspects can comprise a fluid such as saliva, typically as located in an oral cavity (e.g., an oral cavity of a human, primate, dog, or cat). Aqueous conditions can be made to contain sucrose in some aspects through the introduction of a sucrose-containing material (e.g., liquid such as drink and/or solid such as food) to the aqueous conditions. Examples of drinks/beverages and foods that contain sucrose herein can be any as generally disclosed in Int. Patent Appl. Publ. No. WO2023/55902 or U.S. Patent Appl. Publ. No. 2017/0218093, 2022/0322685, or 2021/0282422, for example, which are incorporated herein by reference. When the aqueous conditions are in the mouth/oral cavity, such introduction of sucrose-containing material typically can be via drinking and/or chewing/masticating. When the aqueous conditions are in the oral cavity (mouth), step (b) of a bacterial cell anti-adhesion method can be stated as being performed in the oral cavity.

The aqueous conditions in step (b) of a bacterial cell anti-adhesion method can contain a suitable amount of dextran herein that inhibits adhesion and/or colonization of a bacterial cell. In some aspects, the concentration of dextran in the aqueous conditions is about, or at least about, 0.001, 0.002, 0.003, 0.005, 0.006, 0.008, 0.01, 0.03, 0.05, 0.075, 0.1, 0.25, 0.5, 1, 2, 2.5, 3, 4, 5, 0.003-5, 0.003-4, 0.003-3, 0.003-2.5, 0.003-2, 0.003-1.5, 0.003-1, 0.003-0.5, 0.003-0.1, 0.003-0.05, 0.003-0.01, 0.003-0.008, 0.005-0.5, 0.005-0.1, 0.005-0.05, 0.005-0.01, 0.005-0.008, 0.03-5, 0.03-4, 0.03-3, 0.03-2.5, 0.05-5, 0.05-4, 0.05-3, 0.05-2.5, 0.1-5, 0.1-4, 0.1-3, 0.1-2.5, 0.25-5, 0.25-4, 0.25-3, or 0.25-2.5 g/L. The concentration of the dextran as provided in step (a) of a bacterial cell anti-adhesion method (e.g., as comprised in an oral care product herein) typically is at a level such that any of the foregoing dextran concentrations in the aqueous conditions of step (b) can be met.

The temperature of the aqueous conditions in step (b) of a bacterial cell anti-adhesion method can be about, at least about, or less than about, 15, 20, 25, 30, 35, 37, 40, 42, 45, 50, 15-50, 15-45, 15-40, 15-35, 20-50, 20-45, 20-40, 20-35, 25-50, 25-45, 25-40, 25-35, 30-50, 30-45, 30-40, 30-35, 35-50, 35-45, 35-40, or 36-38° C., for example. Aqueous conditions in some aspects can have a pH of about 4.0, 4.5, 5.0, 5.5, 6.0, 6.5, 7.0, 7.5, 8.0, 8.5, 9.0, or 6.0-8.0, for example. It would be understood that, in aspects in which step (b) of a bacterial cell anti-adhesion method is performed in the oral cavity, the temperature and/or pH of the aqueous conditions can be affected by the temperature and/or pH of a beverage or food that is introduced to the oral cavity and optionally mixed with saliva and/or any pre-existing matter (e.g., beverage/food).

The amount of time used for step (b) of a bacterial cell anti-adhesion method is that time which is suitable for inhibiting adhesion and/or colonization of a bacterial cell herein. In some aspects, the amount of time can be about, or at least about, 5, 10, 15, 20, 25, 30, 40, 50, 60, 90, 120, 150, 180, 5-30, 5-20, 10-30, or 10-20 seconds, or 4, 5, 6, 8, 10, 4-10, 4-8, or 4-6 minutes. In some aspects in which the aqueous conditions comprise sucrose, exposing a bacterial cell to dextran herein can be commenced at about the same time as exposing the bacterial cell to sucrose, or after the bacterial cell has been exposed to sucrose (e.g., after about 1, 5, 10, 15 20, 30, 40, 50, 60, 90, 120, 150, 180, 1-60, 1-30, 1-15, or 1-10 minutes). In aspects in which sucrose is borne from a beverage or food, any of the forgoing time periods can optionally be characterized to correspond with the time following the completion of a mastication and/or imbibing event (e.g., completion of eating a meal/snack, and/or completion of drinking a beverage).

Step (b) of a bacterial cell anti-adhesion method herein (i) inhibits adhesion of a bacterial cell (e.g., adhesion to a surface and/or to another bacterial cell), and/or (ii) inhibits colonization by the bacterial cell (e.g., inhibits biofilm formation by the bacterial cell). Such inhibition ([i] or [ii]) can be by about, or at least about, 10%, 25%, 50%, 75%, 90%, 95%, 99%, or 100%, as compared to the level of inhibition that would have occurred by following the method with all the same parameters except for including the dextran (i.e., as compared to a suitable control). Inhibition of bacterial cell adhesion and/or colonization in some aspects can be measured using any suitable methodology, such as a procedure disclosed in the below Examples. In some aspects, inhibiting adhesion and/or biofilm formation or any other form of surface colonization by bacteria can be achieved by targeting bacterial cells that are planktonic and/or that have settled on a surface. In some aspects in which the method is conducted in an oral cavity using a dental care product/composition herein, it is contemplated that inhibition of adhesion and/or colonization can manifest, at least in part, as a reduction in the occurrence of dental plaque and/or dental caries (cavities). Such plaque reduction and/or dental caries reduction can be by about, or at least about, 10%, 25%, 50%, 75%, 90%, 95%, 99%, or 100%, for example, as compared to the occurrence of dental plaque and/or dental caries that would have occurred if dextran herein was not included in the dental care product (where all other ingredients and application conditions are the same). While not being held to any particular theory, it is contemplated that bacterial cell adhesion inhibition by dextran treatment herein can be due to inhibiting extracellular matrix (ECM) formation by bacteria.

A surface to which a bacterial cell herein can adhere can be an “oral surface”, for example, which encompasses any soft or hard surface within the oral cavity including surfaces of the tongue, hard and soft palate, buccal mucosa, gums and dental surfaces. A surface to which a bacterial cell can adhere can be a tooth surface, for example, such as a mesial, distal, incisal, occlusal, lingual, facial, or buccal tooth surface.

A bacterial cell anti-adhesion method of the present disclosure can target the adhesion of one, two, three, four, or more, or all, species of bacteria that may be present in a bacterial population being treated with dextran herein. In some aspects, a bacterial cell herein requires sucrose (i.e., is sucrose-dependent) for the cell to engage in bacterial cell adhesion and/or colonization. A sucrose-dependent bacterial cell can express at least one sucrase enzyme (e.g., a dextransucrase and/or an alpha-1,3-glucan sucrase), for example; this enzyme expression is typically endogenous/native to the cell. In some aspects, such as those in which step (b) of a bacterial cell anti-adhesion method is performed in an oral cavity, a bacterial cell is cariogenic (i.e., can form a tooth cavity) and/or can produce oral plaque; typically, a population of cariogenic bacterial cells herein is what can form a dental cavity and/or plaque.

A bacterial cell in some aspects is an oral bacterial cell. In some aspects, a bacterial cell can be of the genus Streptococcus, Leuconostoc, or Lactobacillus. Examples of Streptococcus species herein include S. mutans, S. sobrinus, S. sanguinis [S. sanguis], S. salivarius, S. downei, S. dentirousetti, S. oralis, S. criceti, S. gordonii, S. faecalis, S. mitis, and S. gallolyticus, or an oral Streptococcus species. Examples of Leuconostoc species herein include L. mesenteroides, L. pseudomesenteroides, and L. citreum, or an oral Leuconostoc species. Examples of Lactobacillus species herein include L. reuteri, L. acidophilus, L. paracasei, L. rhamnosus, L. casei and L. fermentum, or an oral Lactobacillus species. In some aspects, a bacterial cell herein is not an oral bacterial cell.

A bacterial cell herein can be bound to (or associated with, or complexed to) a dextran molecule(s) of the present disclosure, for example. Such a bacterial cell can be planktonic in some aspects. A planktonic bacterial cell that is bound to dextran herein is contemplated to be able to remain in a planktonic state, even in the presence of sucrose, for a period of time that is about, or at least about, 10%, 25%, 50%, 100%, 250%, 500%, or 1000% longer than a planktonic cell that is not bound to the dextran (but otherwise same conditions/parameters). Such a bacterial cell can be produced by treating a bacterial cell herein with dextran using any method/process as presently disclosed, for example. Such a bacterial cell is contemplated to be unable (or less able) to contribute to biofilm formation, for example, and/or, if present in an oral cavity, such a bacterial cell is contemplated to be able to remain in a planktonic state for a suitable amount of time that allows for clearing the cell from the oral cavity (e.g., by swallowing, rinsing, cleansing/washing, or other activity that removes/replaces fluid in an oral cavity).

In some aspects, a dextran provided in step (a) can be comprised in, or on (e.g., impregnated with), an oral care composition/product. Examples of oral care compositions/products herein include dentifrices (e.g., toothpaste, powder), tooth gel, mouthwash, mouth rinse, anti-plaque rinse, fluoride rinse, chewing gum, edible/dissolvable strips or other solids (e.g., mints), lozenges, dental floss, dental picks/sticks, teeth whitening product (e.g., wash or strip), or any other composition/product that can provide some form of oral care (e.g., treatment or prevention of cavities [dental caries], gingivitis, plaque, tartar, and/or periodontal disease; breath freshening [e.g., halitosis treatment]; teeth whitening; denture or oral prosthesis care). An oral care composition can be in the form of a liquid, paste, gel, foam, gum, or solid/dry solid (e.g., powder, dissolvable product), for example. An oral care composition can also be for treating an oral surface, which encompasses any soft or hard surface within the oral cavity including surfaces of the tongue, hard and soft palate, buccal mucosa, gums and dental surfaces. A dental surface herein is a surface of a natural tooth or a hard surface of artificial dentition including a crown, cap, filling, bridge, denture, or dental implant, for example.

An oral care composition in some aspects can comprise one or more thickening agents and/or dispersion agents that may be useful to impart a desired consistency and/or mouth feel to the composition. Examples of thickening and/or dispersion agents include carboxyvinyl polymer, carrageenan (e.g., L-carrageenan), natural gum (e.g., karaya, xanthan, gum arabic, tragacanth), colloidal magnesium aluminum silicate, alpha-1,3-glucan ether, and colloidal silica. Yet, in some aspects, a water-soluble alpha-glucan as presently disclosed can serve to thicken (i.e., provide or add viscosity to, or, serve as a rheology modifier of) an oral care composition or any other aqueous composition/product herein. An aqueous composition herein can have a viscosity of about, at least about, or less than about, 1, 5, 10, 50, 100, 500, 1000, 5000, 10000, 50000, 100000, 1-10, 1-5, 1-3, 3-10, 3-5, 50000-100000, or 70000-100000 centipoise (cps), for example. Viscosity can be as measured with an aqueous composition herein at any temperature between about 3° C. to about 80° C., for example (e.g., 4-30° C., 15-30° C., 15-25° C.). Viscosity typically is as measured at atmospheric pressure (about 760 torr) or a pressure that is ±10% thereof. Viscosity can be measured using a viscometer or rheometer, for example, and can optionally be as measured at a shear rate (rotational shear rate) of about 0.1, 0.5, 1.0, 5, 10, 50, 100, 500, 1000, 0.1-500, 0.1-100, 1.0-500, 1.0-1000, or 1.0-100 s−1 (1/s), or about 5, 10, 20, 25, 50, 100, 200, or 250 rpm (revolutions per minute), for example. An aqueous composition herein comprising an aqueous-soluble alpha-glucan can have a viscosity (at any given shear rate) that is increased (enhanced) (e.g., about, or at least about, 1.25, 1.5, 2, 2.5, 3, 5, or 10 times higher) as compared to the viscosity the aqueous composition would have if it did not comprise the water-soluble alpha-glucan.

An oral care composition herein may be a toothpaste or other dentifrice, for example. Such compositions, as well as any other oral care composition herein, can additionally comprise, without limitation, one or more of an anticaries agent, antimicrobial or antibacterial agent, anticalculus or tartar control agent, surfactant, abrasive, pH-modifying agent, foam modulator, humectant, flavorant, sweetener, pigment/colorant, whitening agent, and/or other suitable components.

An anticaries agent herein can be an orally acceptable source of fluoride ions. Suitable sources of fluoride ions include fluoride (e.g., sodium fluoride, stannous fluoride), monofluorophosphate and fluorosilicate salts as well as amine fluorides, including olaflur (N′-octadecyltrimethylendiamine-N,N,N′-tris(2-ethanol)-dihydrofluoride), for example. An anticaries agent can be present in an amount providing a total of about 100-20000, 200-5000, or 500-2500 ppm fluoride ions to the composition, for example. In oral care compositions in which sodium fluoride is the sole source of fluoride ions, an amount of about 0.01-5.0, 0.05-1.0, or 0.1-0.5 wt % sodium fluoride can be present in the composition, for example.

An antimicrobial or antibacterial agent suitable for use in an oral care composition herein includes, for example, phenolic compounds (e.g., 4-allylcatechol; p-hydroxybenzoic acid esters such as benzylparaben, butylparaben, ethylparaben, methylparaben and propylparaben; 2-benzylphenol; butylated hydroxyanisole; butylated hydroxytoluene; capsaicin; carvacrol; creosol; eugenol; guaiacol; halogenated bisphenolics such as hexachlorophene and bromochlorophene; 4-hexylresorcinol; 8-hydroxyquinoline and salts thereof; salicylic acid esters such as menthyl salicylate, methyl salicylate and phenyl salicylate; phenol; pyrocatechol; salicylanilide; thymol; halogenated diphenylether compounds such as triclosan and triclosan monophosphate), copper (II) compounds (e.g., copper (II) chloride, fluoride, sulfate and hydroxide), zinc ion sources (e.g., zinc acetate, citrate, gluconate, glycinate, oxide, and sulfate), phthalic acid and salts thereof (e.g., magnesium monopotassium phthalate), hexetidine, octenidine, sanguinarine, benzalkonium chloride, domiphen bromide, alkylpyridinium chlorides (e.g. cetylpyridinium chloride, tetradecylpyridinium chloride, N-tetradecyl-4-ethylpyridinium chloride), iodine, sulfonamides, bisbiguanides (e.g., alexidine, chlorhexidine, chlorhexidine digluconate), piperidino derivatives (e.g., delmopinol, octapinol), magnolia extract, grapeseed extract, rosemary extract, menthol, geraniol, citral, eucalyptol, antibiotics (e.g., augmentin, amoxicillin, tetracycline, doxycycline, minocycline, metronidazole, neomycin, kanamycin, clindamycin), and/or any antibacterial agents disclosed in U.S. Pat. No. 5,776,435, which is incorporated herein by reference. One or more antimicrobial agents can optionally be present at about 0.01-10 wt % (e.g., 0.1-3 wt %), for example, in an oral care composition herein.

Examples of an anticalculus or tartar control agent suitable for use in an oral care composition herein include phosphates and polyphosphates (e.g., pyrophosphates), polyaminopropanesulfonic acid (AMPS), zinc citrate trihydrate, polypeptides (e.g., polyaspartic and polyglutamic acids), polyolefin sulfonates, polyolefin phosphates, diphosphonates (e.g.,azacycloalkane-2,2-diphosphonates such as azacycloheptane-2,2-diphosphonic acid), N-methyl azacyclopentane-2,3-diphosphonic acid, ethane-1-hydroxy-1,1-diphosphonic acid (EHDP), ethane-1-amino-1, 1-diphosphonate, and/or phosphonoalkane carboxylic acids and salts thereof (e.g., their alkali metal and ammonium salts). Useful inorganic phosphate and polyphosphate salts include, for example, monobasic, dibasic and tribasic sodium phosphates, sodium tripolyphosphate, tetrapolyphosphate, mono-, di-, tri- and tetra-sodium pyrophosphates, disodium dihydrogen pyrophosphate, sodium trimetaphosphate, sodium hexametaphosphate, or any of these in which sodium is replaced by potassium or ammonium. Other useful anticalculus agents in some aspects include anionic polycarboxylate polymers (e.g., polymers or copolymers of acrylic acid, methacrylic, and maleic anhydride such as polyvinyl methyl ether/maleic anhydride copolymers). Still other useful anticalculus agents include sequestering agents such as hydroxycarboxylic acids (e.g., citric, fumaric, malic, glutaric and oxalic acids and salts thereof) and aminopolycarboxylic acids (e.g., EDTA). One or more anticalculus or tartar control agents can optionally be present at about 0.01-50 wt % (e.g., about 0.05-25 or 0.1-15 wt %), for example, in an oral care composition herein.

A surfactant suitable for use in an oral care composition herein may be anionic, non-ionic, or amphoteric, for example. Suitable anionic surfactants include, without limitation, water-soluble salts of C₈₋₂₀ alkyl sulfates, sulfonated monoglycerides of C₈₋₂₀ fatty acids, sarcosinates, and taurates. Examples of anionic surfactants include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable non-ionic surfactants include, without limitation, poloxamers, polyoxyethylene sorbitan esters, fatty alcohol ethoxylates, alkylphenol ethoxylates, tertiary amine oxides, tertiary phosphine oxides, and dialkyl sulfoxides. Suitable amphoteric surfactants include, without limitation, derivatives of C₈₋₂₀ aliphatic secondary and tertiary amines having an anionic group such as a carboxylate, sulfate, sulfonate, phosphate or phosphonate. An example of a suitable amphoteric surfactant is cocoamidopropyl betaine. One or more surfactants can optionally be present in a total amount of about 0.01-10 wt % (e.g., about 0.05-5.0 or 0.1-2.0 wt %), for example, in an oral care composition herein.

An abrasive suitable for use in an oral care composition herein may include, for example, silica (e.g., silica gel, hydrated silica, precipitated silica), alumina, insoluble phosphates, calcium carbonate, and resinous abrasives (e.g., a urea-formaldehyde condensation product). Examples of insoluble phosphates useful as abrasives herein are orthophosphates, polymetaphosphates and pyrophosphates, and include dicalcium orthophosphate dihydrate, calcium pyrophosphate, beta-calcium pyrophosphate, tricalcium phosphate, calcium polymetaphosphate and insoluble sodium polymetaphosphate. One or more abrasives can optionally be present in a total amount of about 5-70 wt % (e.g., about 10-56 or 15-30 wt %), for example, in an oral care composition herein. The average particle size of an abrasive in some aspects can be about 0.1-30 microns (e.g., about 1-20 or 5-15 microns).

An oral care composition herein can comprise at least one pH-modifying agent, for example. Such an agent typically is selected to acidify, make more basic, or buffer the pH of a composition to a pH range of about 2-10 (e.g., pH ranging from about 2-8, 3-9, 4-8, 5-7, 6-10, or 7-9). Examples of pH-modifying agents useful herein include, without limitation, carboxylic, phosphoric and sulfonic acids; acid salts (e.g., monosodium citrate, disodium citrate, monosodium malate); alkali metal hydroxides (e.g. sodium hydroxide, carbonates such as sodium carbonate, bicarbonates, sesquicarbonates); borates; silicates; phosphates (e.g., monosodium phosphate, trisodium phosphate, pyrophosphate salts); and imidazole.

A foam modulator suitable for use in an oral care composition herein may be a polyethylene glycol (PEG), for example. High molecular weight PEGs are suitable, for example, including those having an average molecular weight of about 200000-7000000 (e.g., about 500000-5000000 or 1000000-2500000). One or more PEGs can optionally be present in a total amount of about 0.1-10 wt % (e.g. about 0.2-5.0 or 0.25-2.0 wt %), for example, in an oral care composition herein.

An oral care composition in some aspects may comprise at least one humectant. A humectant may be a polyhydric alcohol, for example, such as glycerin, sorbitol, xylitol, or a low molecular weight PEG. Most suitable humectants can also function as a sweetener herein, for example. One or more humectants can optionally be present in a total amount of about 1.0-70 wt % (e.g., about 1.0-50, 2-25, or 5-15 wt %), for example, in an oral care composition herein.

A natural or artificial sweetener can optionally be comprised in an oral care composition herein. Examples of suitable sweeteners include dextrose, sucrose, maltose, dextrin, invert sugar, mannose, xylose, ribose, fructose, levulose, galactose, corn syrup (e.g., high fructose corn syrup or corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, and cyclamates. One or more sweeteners can optionally be present in a total amount of about 0.005-5.0 wt %, for example, in an oral care composition herein.

A natural or artificial flavorant can optionally be comprised in an oral care composition herein. Examples of suitable flavorants include vanillin; sage; marjoram; parsley oil; spearmint oil; cinnamon oil; oil of wintergreen (methylsalicylate); peppermint oil; clove oil; bay oil; anise oil; eucalyptus oil; citrus oils; fruit oils; essences such as those derived from lemon, orange, lime, grapefruit, apricot, banana, grape, apple, strawberry, cherry, or pineapple; bean- and nut-derived flavors such as coffee, cocoa, cola, peanut, or almond; and adsorbed and encapsulated flavorants. Also encompassed within flavorants herein are ingredients that provide fragrance and/or other sensory effect(s) in the mouth, including cooling or warming effects. Such ingredients include, without limitation, menthol, menthyl acetate, menthyl lactate, camphor, eucalyptus oil, eucalyptol, anethole, eugenol, cassia, oxanone, Irisone®, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), and menthone glycerol acetal (MGA). One or more flavorants can optionally be present in a total amount of about 0.01-5.0 wt % (e.g., about 0.1-2.5 wt %), for example, in an oral care composition herein.

An oral care composition in some aspects can comprise at least one bicarbonate salt. Any orally acceptable bicarbonate can be used, for example, including an alkali metal bicarbonate such as sodium or potassium bicarbonate, or ammonium bicarbonate. One or more bicarbonate salts can optionally be present in a total amount of about 0.1-50 wt % (e.g., about 1-20 wt %), for example, in an oral care composition herein.

An oral care composition in some aspects can comprise at least one whitening agent and/or colorant. A suitable whitening agent is a peroxide compound, for example, such as disclosed in U.S. Pat. No. 8,540,971, which is incorporated herein by reference. Suitable colorants herein include pigments, dyes, lakes and agents imparting a particular luster or reflectivity such as pearling agents, for example. Specific examples of colorants useful herein include talc; mica; magnesium carbonate; calcium carbonate; magnesium silicate; magnesium aluminum silicate; silica; titanium dioxide; zinc oxide; red, yellow, brown and black iron oxides; ferric ammonium ferrocyanide; manganese violet; ultramarine; titaniated mica; and bismuth oxychloride. One or more colorants can optionally be present in a total amount of about 0.001-20 wt % (e.g., about 0.01-10 or 0.1-5.0 wt %), for example, in an oral care composition herein.

Additional components that can optionally be included in an oral care composition herein include one or more isolated enzymes (active enzymes), vitamins, and anti-adhesion agents, for example. Examples of vitamins useful herein include vitamin C, vitamin E, vitamin B5, and folic acid. Examples of suitable anti-adhesion agents include solbrol, ficin, and quorum-sensing inhibitors. Examples of suitable enzymes include proteases, cellulases, hemicellulases, peroxidases, lactoperoxidases, lipolytic enzymes (e.g., metallolipolytic enzymes), xylanases, lipases, phospholipases, esterases (e.g., arylesterase, polyesterase), perhydrolases, glycohydrolases, cutinases, pectinases, pectate lyases, mannanases, keratinases, reductases, oxidases (e.g., choline oxidase, glucose oxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, arabinofuranosidases, phytases, isomerases, transferases, nucleases (e.g., deoxyribonuclease [DNase]), mutanases, and amylases (e.g., alpha-amylase, beta-amylase). In some aspects, enzymes can include a nuclease such as a DNase and a glycohydrolase such as a mutanase. If an enzyme(s) is included, it may be comprised in a composition herein at about 0.0001-0.1 wt % (e.g., 0.01-0.03 wt %) active enzyme (e.g., calculated as pure enzyme protein), for example.

In some aspects, an oral care composition/product/formulation that can be adapted for use herein can be as disclosed in any of U.S. Patent Appl. Publ. Nos. 2006/0134025, 2002/0022006, 2008/0057007, 2010/0135930, 2003/0044359, 2006/0088482, 2007/0014740, 2008/0187498, 2013/0344120, 2013/0280180, or 2022/0257484, or U.S. Pat. Nos. 4,528,181, 4,575,456, 5,624,906, 5,658,553, 5,833,958, 6,143,281, 6,379,654, 6,506,366, 3,678,154, 3,535,421, 3,862,307, 3,988,433, 4,022,880, 4,083,955, 4,138,477, 5,004,597, 5,534,243, 4,992,276, 4,945,087, 4,923,685, 4,839,158, 4,824,661, 4,719,100, 4,716,035, 4,606,911, 4,525,343, 4,323,551, 4,312,889, 4,152,418, 4,082,841, 3,988,433, 3,954,962, 3,560,608, 6,723,305, 9,241,885, 9,084,902, 9,877,930, 9,913,783, 10039697, 10149806, 10226414, 10231910, or 9884008, each of which are incorporated herein by reference. It is contemplated that numerous commercially available oral care compositions/products/formulations can be adapted accordingly for use herein. Some examples of commercially available oral care compositions/products/formulations include (i) toothpastes such as AIM, AQUAFRESH, CLOSE-UP, COLGATE (COLGATE TOTAL), CREST, ORAL-B, PEPSODENT, REMBRANDT, SENSODYNE, TOM'S OF MAINE, YUNNAN BAIYAO, DARLIE, ZHONG HUA, LION, DABUR MESWAK, DABUR RED PASTE, and BENTODENT, (ii) mouthwashes/rinses such as ACT, LISTERINE, CREST PRO-HEALTH, BIOTENE, COLGATE TOTAL, SCOPE, THERABREATH, and HELLO, (iii) dissolvable strips and other dissolvable solids such as LISTERINE POCKETPAKS (strips), LISTERINE READY! TABS (chewables), ACT DRY MOUTH (lozenges), ICE BREAKERS (mints), ALTOIDS (mints), BREATH SAVERS (mints), CERTS (mints), and TIC TAC (mints/other), and (iv) tooth floss and picks/sticks such as REACH, ORAL-B, GLIDE, GUM, DENTEK, STIM-U-DENT, and PLACKERS.

An oral care composition/product of the present disclosure typically has increased activity to:
(i) inhibit adhesion of a bacterial cell (e.g., adhesion to a surface and/or to another bacterial cell),
(ii) inhibit colonization by a bacterial cell (e.g., inhibit biofilm formation by the bacterial cell), and/or
(iii) inhibit formation of extracellular matrix (ECM) by the bacterial cell, wherein this increased inhibitory activity of (i), (ii), or (iii) is as compared to the activity (inhibitory activity of i, ii, or iii) of a reference oral care composition/product that does not comprise a dextran herein (e.g., the reference oral care composition/product only differs from the forgoing oral care composition/product by lacking the dextran).

As described herein, dextran can be comprised in an oral care composition/product. Yet, in some aspects, dextran herein can be in the form of, and/or comprised in, a household care (home care) product, personal care product, industrial product, ingestible product (e.g., food product), pharmaceutical product, or medical product, for example, such as described in any of U.S. Patent Appl. Publ. Nos. 2018/0022834, 2018/0237816, 2018/0230241, 20180079832, 2016/0311935, 2016/0304629, 2015/0232785, 2015/0368594, 2015/0368595, 2016/0122445, 2019/0202942, or 2019/0309096, or International Patent Appl. Publ. No. WO2016/133734, which are each incorporated herein by reference. In some aspects, a composition/product herein can comprise at least one component/ingredient of a household care product, personal care product, industrial product, ingestible product (e.g., food product), pharmaceutical product, or medical product as disclosed in any of the foregoing publications and/or as presently disclosed.

Merely as an example of dextran that can be comprised in a composition/product such as a household care product, personal care product, industrial product, ingestible product (e.g., food product), pharmaceutical product, or medical product, a dextran can be one as described herein as produced in an isolated enzymatic reaction that comprises at least one dextransucrase and at least one alpha-1,2-branching enzyme (also including at least water and sucrose). Merely as another example, a dextran can be one as described herein that is a grafted dextran polymer.

Non-limiting examples of compositions/products and methods/processes disclosed herein include:

1. A method (process) of inhibiting (or controlling, preventing, or blocking) at least one bacterial cell, the method comprising: (a) providing a water-soluble alpha-glucan, wherein at least about 30% of the glycosidic linkages of the water-soluble alpha-glucan are alpha-1,6 linkages (i.e., the alpha-glucan is alpha-1,6-glucan or dextran), and (b) contacting at least one bacterial cell in aqueous conditions (an aqueous setting) with the water-soluble alpha-glucan (and/or bringing the alpha-glucan into close proximity of the bacterial cell), wherein the contacting: (i) inhibits adhesion of the bacterial cell (e.g., adhesion to a surface and/or to another bacterial cell), and/or (ii) inhibits colonization by the bacterial cell (e.g., inhibits biofilm formation by the bacterial cell, or disperses a biofilm or colony containing the bacterial cell) (and/or [iii] inhibits formation of extracellular matrix by the bacterial cell).
2. The method of embodiment 1, wherein: (i) the weight-average molecular weight of the water-soluble alpha-glucan is less than about 300 kDa (e.g., less than about 250 kDa or less than about 225 kDa), and/or (ii) the weight-average molecular weight of the water-soluble alpha-glucan is greater than about 5 kDa (e.g., greater than about 8 kDa or greater than about 10 kDa).
3. The method of embodiment 1 or 2, wherein at least about 50% of the glycosidic linkages of the water-soluble alpha-glucan are alpha-1,6 linkages.
4. The method of embodiment 1, 2, or 3, wherein about 100% of the glycosidic linkages of the water-soluble alpha-glucan are alpha-1,6 linkages.
5. The method of embodiment 1, 2, or 3, wherein the water-soluble alpha-glucan comprises one or more alpha-1,2 branches, optionally wherein at least one of the branches comprises a side chain (meaning a side chain to the backbone from which the at least one branch originates; i.e., the at least one branch is the side chain), wherein at least about 50% of the glycosidic linkages of the side chain are alpha-1,6 linkages (e.g., a grafted dextran polymer herein).
5a. The method of embodiment 1, 2, 3, or 4, wherein the water-soluble alpha-glucan comprises one or more alpha-1,3 branches, optionally wherein at least one of the branches comprises a side chain (meaning a side chain to the backbone from which the at least one branch originates; i.e., the at least one branch is the side chain), wherein at least about 50% of the glycosidic linkages of the side chain are alpha-1,6 linkages (e.g., a grafted dextran polymer herein).
6. The method of embodiment 1, 2, 3, 4, 5, or 5a, wherein the water-soluble alpha-glucan comprises at least about 1% alpha-1,2 branches.
7. The method of embodiment 1, 2, 3, 4, 5, 5a, or 6, wherein the water-soluble alpha-glucan comprises at least about 1% alpha-1,3 branches.
8. The method of embodiment 1, 2, 3, 4, 5, 5a, 6, or 7, wherein the water-soluble alpha-glucan was produced in an isolated reaction comprising at least: (i) water, (ii) sucrose, and (iii) at least one isolated glucosyltransferase enzyme that synthesizes alpha-1,6-glucan (i.e., a dextransucrase), wherein at least about 30% of the glycosidic linkages of the alpha-1,6-glucan are alpha-1,6 linkages.
9. The method of embodiment 8, wherein only one or two of the at least one isolated glucosyltransferase enzyme (one or two different glucosyltransferases) is used.
10. The method of embodiment 8 or 9, wherein the isolated reaction further comprises: (iv) at least one isolated glucosyltransferase enzyme that forms at least one alpha-1,2 branch from the alpha-1,6-glucan (i.e., a branching sucrase).
10a. The method of embodiment 8, 9, or 10, wherein the isolated reaction further comprises: at least one isolated glucosyltransferase enzyme that forms at least one alpha-1,3 branch from the alpha-1,6-glucan (i.e., a branching sucrase).
11. The method of embodiment 1, 2, 3, 4, 5, 5a, 6, 7, 8, 9, 10, or 10a, wherein the aqueous conditions comprise sucrose.
12. The method of embodiment 1, 2, 3, 4, 5, 5a, 6, 7, 8, 9, 10, 10a, or 11, wherein the bacterial cell requires sucrose (i.e., is sucrose-dependent) for the adhesion and/or colonization, typically wherein the bacterial cell expresses at least one sucrase enzyme.
13. The method of embodiment 1, 2, 3, 4, 5, 5a, 6, 7, 8, 9, 10, 10a, 11, or 12, wherein the bacterial cell is of (is a species of) the genus Streptococcus, Leuconostoc, or Lactobacillus.
14. The method of embodiment 1, 2, 3, 4, 5, 5a, 6, 7, 8, 9, 10, 10a, 11, 12, or 13, wherein the bacterial cell is cariogenic and/or can produce oral plaque.
15. The method of embodiment 1, 2, 3, 4, 5, 5a, 6, 7, 8, 9, 10, 10a, 11, 12, 13, or 14, wherein contacting step (b) is performed in an oral cavity (typically that of a mammal such as a human, primate, dog, or cat).
16. The method of embodiment 1, 2, 3, 4, 5, 5a, 6, 7, 8, 9, 10, 10a, 11, 12, 13, 14, or 15, wherein the water-soluble alpha-glucan provided in step (a) is comprised in, or on (e.g., impregnated on), an oral care composition/product (e.g., dentifrice [toothpaste or powder], tooth gel, mouthwash, mouth rinse, anti-plaque rinse, fluoride rinse, chewing gum, edible/dissolvable strip or other solid (e.g., mint), lozenge, dental floss, dental pick/stick, or teeth whitening product [e.g., wash or strip]).
17. The method of embodiment 1, 2, 3, 4, 5, 5a, 6, 7, 8, 9, 10, 10a, 11, 12, 13, 14, 15, or 16, wherein the concentration of the water-soluble alpha-glucan in the aqueous conditions is at least about 0.003 g/L.
18. An oral care composition (product) comprising a water-soluble alpha-glucan, wherein at least about 30% of the glycosidic linkages of the water-soluble alpha-glucan are alpha-1,6 linkages (i.e., the alpha-glucan is alpha-1,6-glucan or dextran) (e.g., water-soluble alpha-glucan can be according to embodiment 1, 2, 3, 4, 5, 5a, 6, 7, 8, 9, 10, or 10a), wherein the oral care composition/product has increased activity to: (i) inhibit adhesion of a bacterial cell (e.g., adhesion to a surface and/or to another bacterial cell), and/or (ii) inhibit colonization by a bacterial cell (e.g., inhibit biofilm formation by the bacterial cell, or disperse a biofilm or colony containing the bacterial cell) (and/or [iii] inhibit formation of extracellular matrix by the bacterial cell), wherein the increased activity is as compared to the activity (inhibitory activity of [i], [ii], or [iii]) of a reference/control oral care composition/product that does not comprise the water-soluble alpha-glucan (e.g., the reference oral care composition/product only differs from the first-recited oral care composition/product by lacking the water-soluble alpha-glucan).
19. A composition/product (e.g., household care product, personal care product, industrial product, ingestible product [e.g., food product], pharmaceutical product, or medical product) comprising a water-soluble alpha-glucan according to embodiment 1, 2, 3, 4, 5, 5a, 6, 7, 8, 9, 10, or 10a.

### Examples

The present disclosure is further exemplified in the following Examples. It should be understood that these Examples, while indicating certain aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### Production of Water-Soluble Alpha-Glucan Polymer

A. Sucrose (2 g) was combined with 5 mL of 100 mM sodium acetate, 10 μL of 1M calcium chloride, and Milli-Q® water to a final volume of 10 mL in two 15-mL tubes. GTF 6831 (200 μL of 53 U/mL) alone, or both GTF 6831 (200 μL of 53 U/mL) and GTF 9905 (200 μL 10 U/mL), were added to start each reaction. The two reactions were placed at 37° C. for five hours with end-over-end mixing.

Both GTFs 6831 and 9905 were as disclosed in U.S. Patent Appl. Publ. No. 2018/0282385, which is incorporated herein by reference. GTF 6831 (a dextransucrase) produces soluble alpha-glucan having about 100% alpha-1,6 linkages (i.e., a dextran) with a weight-average molecular weight (Mw) generally less than about 200 kDa. GTF 9905 is a branching sucrase that produces alpha-1,2 branches from a dextran substrate (dextran backbone); each branch as added by GTF 9905 typically is of a single (pendant) alpha-1,2-linked glucosyl unit. It is believed that, in the above reaction containing both GTFs 6831 and 9905, an alpha-1,2 branch can be extended by GTF 6831 to effectively make a dextran branch (or side group). It is possible that this branching process can iteratively occur to produce a dextran with branch-on-branch structure.

B. Another pair of soluble alpha-glucan synthesis reactions was also performed as follows. Sucrose (1 g) was combined with 2.5 mL of 100 mM sodium acetate, 5 μL of 1 M calcium chloride, and Milli-Q® water to a final volume of 5 mL in two 15-mL tubes. GTF 6831 (100 μL of 53 U/mL) or GTF 0768 (100 μL of 1.8 U/mL) were then added to start each respective reaction. The reactions were placed for about four hours at 30° C. with end-over-end mixing.

GTF 0768 (a dextransucrase) produces soluble branched alpha-glucan with alpha-1,6 linkages and high Mw (generally over 1 million Daltons) (U.S. Patent Appl. Publ. No. 2016/0122445, which is incorporated herein by reference).

### Ethanol Precipitation of Alpha-Glucan Polymer

The above reactions, upon completion, were individually transferred to 50-mL tubes and chilled on ice. Ice-cold ethanol (200 proof, 10 or 20 mL) was then gradually added to each tube with constant gentle mixing. The tubes were centrifuged at 3500 rpm for 15 minutes and ethanol supernatant was removed by aspiration. Each of the pellets was then suspended and dissolved in Milli-Q® water (10 mL) at 95° C. Any undissolved polymer was removed by centrifugation.

### Maltodextrin Control

Maltodextrin (1 g, DE 8-15, Sigma Cat. No. 31410), 5 mL of 100 mM sodium acetate, 10 μL of 1 M calcium chloride, and Milli-Q® water were added to a final volume of 10 mL in a single 50-mL tube. Ice-cold ethanol (200 proof, 10 mL) was then added slowly to the tube while gently mixing. The tube was centrifuged at 3500 rpm for 15 minutes and ethanol supernatant was removed by aspiration. The pellet was suspended and dissolved in Milli-Q® water (10 mL) at 95° C. Any undissolved polymer was removed by centrifugation.

### Biofilm Formation Assay

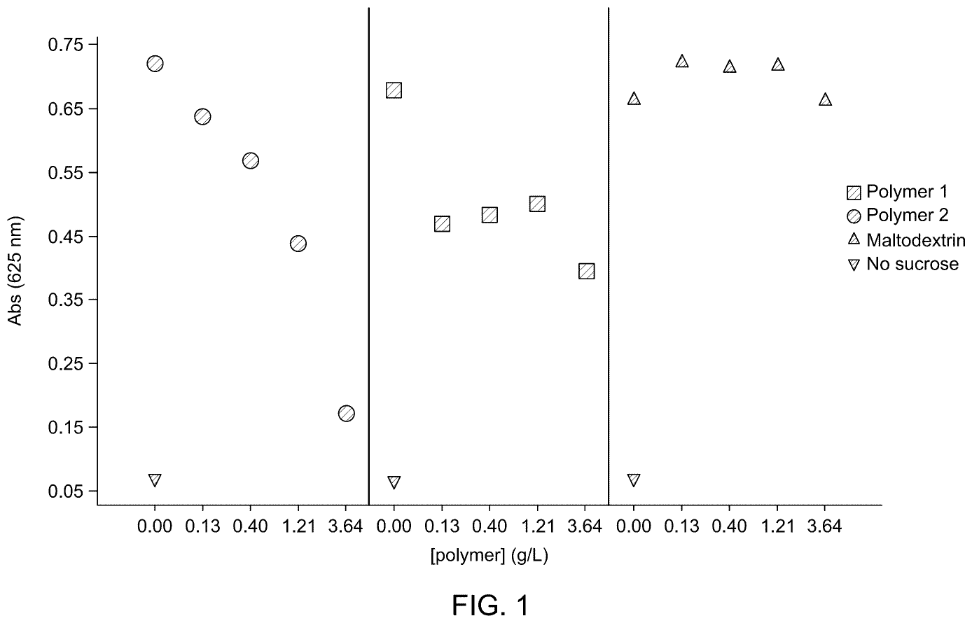

A culture of Streptococcus mutans (American Type Culture Collection [ATCC] No. 25175) was grown overnight in brain heart infusion (BHI) media at 37° C. without shaking. Each biofilm assay was conducted in 24-well microtiter plates (Thermo Scientific 142475). Adherence media (900 μL; Takada et al., Infect. Immunity 50:833-843, incorporated herein by reference) was first added to each well, followed by 100 μL of 20 wt % sucrose, 100 μL of an alpha-glucan polymer solution (0-40 μg/L), and 35 μL of the overnight culture. The polymers that were individually tested were maltodextrin or an alpha-glucan product as prepared in A or B above. Each tested condition (polymer/concentration, control wells) had four replicates in the assay. Control wells in which sucrose was added or not added—each condition without polymer—were included to determine baseline levels of biofilm formation under either condition (plus/minus sucrose). The no-sucrose-added wells were not expected to support biofilm formation. The plates were incubated overnight at 37° C. without shaking. The next day, the liquid from each plate was discarded, and the wells were gently washed three times with 2 mL of phosphate buffered saline (PBS). The plate wells were treated with 2 mL of 0.01% crystal violet staining solution for 30 minutes at room temperature with no shaking, this treatment stained any cells that adhered to the plate as a biofilm. The staining solution was discarded, after which the wells were washed three times with 2 mL of PBS. Any biofilm/cell-bound crystal violet stain was released by the addition of 1 mL of 30% acetic acid to each well. One-hundred μL of the crystal violet/acetic acid solution in each well was then transferred to a 96-well microtiter plate (Corning 9017) containing 100 μL of water per well, and the plate was read at 625 nm. The reported absorbance values (FIG. 1) are the average of the four replicates performed for each tested condition.

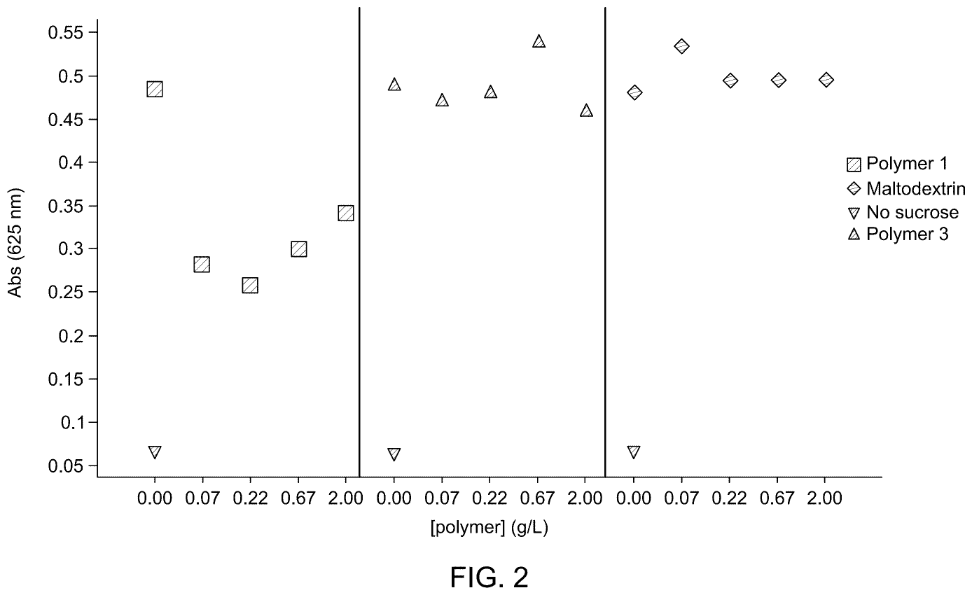

As shown in FIG. 1, including soluble alpha-glucan that was (i) produced by GTF 6831 alone (Polymer 1), or (ii) produced by GTFs 6831 and 9905 together (Polymer 2), in S. mutans cultures inhibited the sucrose-dependent biofilm formation ability of S. mutans cells in a dose-dependent manner. However, treatment with the soluble alpha-glucan, maltodextrin (a type of alpha-1,4-glucan), did not inhibit biofilm formation (FIG. 1). FIG. 2 shows results of another test demonstrating that soluble alpha-glucan produced by GTF 6831 (Polymer 1) inhibited biofilm formation. Interestingly, FIG. 2 also shows that soluble alpha-glucan produced by GTF 0768 (Polymer 3) generally did not inhibit biofilm formation. These results altogether indicate that some soluble alpha-glucans with alpha-1,6 linkages can be useful in controlling adhesion and/or biofilm formation by S. mutans and likely other bacterial species (e.g., cariogenic bacteria) that utilize sucrose to form extracellular matrices (ECM). It was also observed that there was no inhibition of biofilm formation when conducting the assay with 0.08, 0.4, 2.0, or 10.0 g/L xylitol (instead of an alpha-1,6 glucan as above).

Additional dose-dependent assays (performed as above) were conducted to investigate the inhibitory effects of various alpha-1,2-branched alpha-1,6-glucan or alpha-1,3-branched alpha-1,6-glucan compounds on biofilm formation by S. mutans cells (Table 1).

TABLE 1

| Polymer | | MW^{a} | Percent | Concentration | % Biofilm |
|---|---|---|---|---|---|
| Designation | Polymer Description | (kDa) | Branching | (mg/L) | Remaining |
| Polymer-1 | Alpha (1,6) with | 3 | 5% | 27 | 76% |
| | alpha(1,2) branching | | | | |
| Polymer-1 | Alpha (1,6) with | 3 | 5% | 109 | 85% |
| | alpha(1,2) branching | | | | |
| Polymer-1 | Alpha (1,6) with | 3 | 5% | 435 | 78% |
| | alpha(1,2) branching | | | | |
| Polymer-1 | Alpha (1,6) with | 3 | 5% | 1740 | 76% |
| | alpha(1,2) branching | | | | |
| Polymer-2 | Alpha (1,6) with | 3 | 45% | 26 | 88% |
| | alpha(1,2) branching | | | | |
| Polymer-2 | Alpha (1,6) with | 3 | 45% | 104 | 99% |
| | alpha(1,2) branching | | | | |
| Polymer-2 | Alpha (1,6) with | 3 | 45% | 418 | 100% |
| | alpha(1,2) branching | | | | |
| Polymer-2 | Alpha (1,6) with | 3 | 45% | 1670 | 106% |
| | alpha(1,2) branching | | | | |
| Polymer-3 | Alpha (1,6) with | 12 | 40% | 30 | 92% |
| | alpha(1,2) branching | | | | |
| Polymer-3 | Alpha (1,6) with | 12 | 40% | 119 | 68% |
| | alpha(1,2) branching | | | | |
| Polymer-3 | Alpha (1,6) with | 12 | 40% | 475 | 48% |
| | alpha(1,2) branching | | | | |
| Polymer-3 | Alpha (1,6) with | 12 | 40% | 1900 | 40% |
| | alpha(1,2) branching | | | | |
| Polymer-4 | Alpha (1,6) with | 14 | 20% | 28 | 34% |
| | alpha(1,2) branching | | | | |
| Polymer-4 | Alpha (1,6) with | 14 | 20% | 114 | 25% |
| | alpha(1,2) branching | | | | |
| Polymer-4 | Alpha (1,6) with | 14 | 20% | 455 | 21% |
| | alpha(1,2) branching | | | | |
| Polymer-4 | Alpha (1,6) with | 14 | 20% | 1820 | 21% |
| | alpha(1,2) branching | | | | |
| Polymer-5 | Alpha (1,6) with | 40 | 20% | 28 | 55% |
| | alpha(1,2) branching | | | | |
| Polymer-5 | Alpha (1,6) with | 40 | 20% | 114 | 44% |
| | alpha(1,2) branching | | | | |
| Polymer-5 | Alpha (1,6) with | 40 | 20% | 455 | 35% |
| | alpha(1,2) branching | | | | |
| Polymer-5 | Alpha (1,6) with | 40 | 20% | 1820 | 31% |
| | alpha(1,2) branching | | | | |
| Polymer-6 | Alpha (1,6) with | 40 | 40% | 27 | 93% |
| | alpha(1,2) branching | | | | |
| Polymer-6 | Alpha (1,6) with | 40 | 40% | 109 | 99% |
| | alpha(1,2) branching | | | | |
| Polymer-6 | Alpha (1,6) with | 40 | 40% | 438 | 97% |
| | alpha(1,2) branching | | | | |
| Polymer-6 | Alpha (1,6) with | 40 | 40% | 1750 | 101% |
| | alpha(1,2) branching | | | | |
| Polymer-7 | Alpha (1,6) with | 200 | 5% | 29 | 37% |
| | alpha(1,2) branching | | | | |
| Polymer-7 | Alpha (1,6) with | 200 | 5% | 117 | 30% |
| | alpha(1,2) branching | | | | |
| Polymer-7 | Alpha (1,6) with | 200 | 5% | 468 | 27% |
| | alpha(1,2) branching | | | | |
| Polymer-7 | Alpha (1,6) with | 200 | 5% | 1870 | 38% |
| | alpha(1,2) branching | | | | |
| Polymer-8 | Alpha (1,6) with | 200 | 20% | 31 | 57% |
| | alpha(1,2) branching | | | | |
| Polymer-8 | Alpha (1,6) with | 200 | 20% | 126 | 46% |
| | alpha(1,2) branching | | | | |
| Polymer-8 | Alpha (1,6) with | 200 | 20% | 503 | 33% |
| | alpha(1,2) branching | | | | |
| Polymer-8 | Alpha (1,6) with | 200 | 20% | 2010 | 26% |
| | alpha(1,2) branching | | | | |
| Polymer-9 | Alpha (1,6) with | 10 | 15% | 26 | 39% |
| | alpha(1,3) branching | | | | |
| Polymer-9 | Alpha (1,6) with | 10 | 15% | 104 | 24% |
| | alpha(1,3) branching | | | | |
| Polymer-9 | Alpha (1,6) with | 10 | 15% | 415 | 19% |
| | alpha(1,3) branching | | | | |
| Polymer-9 | Alpha (1,6) with | 10 | 15% | 1660 | 24% |
| | alpha(1,3) branching | | | | |
| Polymer-10 | polydextrose | 10 | n/a | 28 | 110% |
| Polymer-10 | polydextrose | 10 | n/a | 112 | 106% |
| Polymer-10 | polydextrose | 10 | n/a | 448 | 103% |
| Polymer-10 | polydextrose | 10 | n/a | 1790 | 107% |

^{a}MW: molecular weight of the alpha-1,6-glucan before having been alpha-1,2- or alpha-1,3-branched.

Additional assays (performed as above) were conducted to investigate the inhibitory effects of various alpha-1,2-branched alpha-1,6-glucan or alpha-1,3-branched alpha-1,6-glucan compounds on biofilm formation by human caries S. mutans isolates (Table 2). Strain ATCC 25175 (S. mutans Clarke) was originally isolated from carious human dentine. S. mutans Ingbritt (serotype c) was originally described by Krasse (1966, Archs. Oral Biol. 11:429, incorporated herein by reference) and has been widely used for studying S. mutans. S. mutans 2366 is an isolate originally identified in a clinical study; details of its origin, isolation, and identification have been described (Söderling et al., 2000, J. Dent. Res. 79:882-887; Söderling et al., 2008, Curr. Microbiol. 56:382-385; Söderling and Hietala-Lenkkeri, 2010, Curr. Microbiol. 60, 25-29; each of which is incorporated herein by reference). Each of the polymers (4, 7 and 9) listed in Table 2 is as listed in Table 1.

TABLE 2

| | Polymer | Concentration | % Biofilm |
|---|---|---|---|
| Strain | Designation | (g/L) | Remaining |
| ATCC 25175 | Polymer 7 | 2 | 39% |
| ATCC 25175 | Polymer 9 | 2 | 14% |
| ATCC 25175 | Polymer 4 | 2 | 41% |
| Ingbritt | Polymer 7 | 2 | 74% |
| Ingbritt | Polymer 9 | 2 | 49% |
| Ingbritt | Polymer 4 | 2 | 58% |
| clinical isolate | Polymer 7 | 2 | 57% |
| 2366 | | | |
| clinical isolate | Polymer 9 | 2 | 32% |
| 2366 | | | |
| clinical isolate | Polymer 4 | 2 | 59% |
| 2366 | | | |

The percent of biofilm remaining as reported for each assay in Tables 1 and 2 was calculated using Equation 1:

$$\% Biofilm Remaining = \frac{( Avg Abs with glucan \& sucrose ) - ( Control 1 )}{( Control 2 ) - ( Control 1 )} \times 100$$

Each data point was the average of four replicates from the same 24-well plate. Control 1 was the background signal from the assay, and it contained no glucan and no sucrose. Control 2 was the maximum biofilm signal, and it contained sucrose but no glucan. Each 24-well plate contained Control 1, Control 2, and four concentrations (Table 1) of glucan. Equation 1 can also be represented as:

$$\% Biofilm Remaining = \frac{\begin{matrix}( Avg Abs with glucan \& sucrose ) - \\ ( Avg Abs without glucan \& sucrose )\end{matrix}}{\begin{matrix}( Avg Abs without glucan \& with sucrose ) - \\ ( Avg Abs without glucan \& sucrose )\end{matrix}} \times 100$$

What is claimed is:

1. A method of inhibiting at least one bacterial cell, the method comprising:
(a) providing a water-soluble alpha-glucan, wherein at least about 30% of the glycosidic linkages of the water-soluble alpha-glucan are alpha-1,6 linkages, and
(b) contacting at least one bacterial cell in aqueous conditions with the water-soluble alpha-glucan, wherein said contacting:
(i) inhibits adhesion of the bacterial cell, and/or
(ii) inhibits colonization by the bacterial cell.

2. The method of claim 1, wherein:
(i) the weight-average molecular weight of the water-soluble alpha-glucan is less than about 300 kDa, and/or
(ii) the weight-average molecular weight of the water-soluble alpha-glucan is greater than about 5 kDa.

3. The method of claim 1, wherein at least about 50% of the glycosidic linkages of the water-soluble alpha-glucan are alpha-1,6 linkages.

4. The method of claim 1, wherein about 100% of the glycosidic linkages of the water-soluble alpha-glucan are alpha-1,6 linkages.

5. The method of claim 1, wherein the water-soluble alpha-glucan comprises one or more alpha-1,2 branches,
optionally wherein at least one of the branches comprises a side chain, wherein at least about 50% of the glycosidic linkages of the side chain are alpha-1,6 linkages.

6. The method of claim 1, wherein the water-soluble alpha-glucan comprises at least about 1% alpha-1,2 branches.

7. The method of claim 1, wherein the water-soluble alpha-glucan comprises at least about 1% alpha-1,3 branches.

8. The method of claim 1, wherein the water-soluble alpha-glucan was produced in an isolated reaction comprising at least:
(i) water,
(ii) sucrose, and
(iii) at least one isolated glucosyltransferase enzyme that synthesizes alpha-1,6-glucan, wherein at least about 30% of the glycosidic linkages of the alpha-1,6-glucan are alpha-1,6 linkages.

9. The method of claim 8, wherein only one or two of said at least one isolated glucosyltransferase enzyme is used.

10. The method of claim 8, wherein said isolated reaction further comprises:
(iv) at least one isolated glucosyltransferase enzyme that forms at least one alpha-1,2 branch from said alpha-1,6-glucan.

11. The method of claim 1, wherein the aqueous conditions comprise sucrose.

12. The method of claim 1, wherein the bacterial cell requires sucrose for said adhesion and/or colonization, typically wherein the bacterial cell expresses at least one sucrase enzyme.

13. The method of claim 1, wherein the bacterial cell is of the genus Streptococcus, Leuconostoc, or Lactobacillus.

14. The method of claim 1, wherein the bacterial cell is cariogenic and/or can produce oral plaque.

15. The method of claim 1, wherein contacting step (b) is performed in an oral cavity.

16. The method of claim 1, wherein the water-soluble alpha-glucan provided in step (a) is comprised in, or on, an oral care composition/product.

17. The method of claim 1, wherein the concentration of the water-soluble alpha-glucan in the aqueous conditions is at least about 0.003 g/L.

18. An oral care composition/product comprising a water-soluble alpha-glucan, wherein at least about 30% of the glycosidic linkages of the water-soluble alpha-glucan are alpha-1,6 linkages, wherein said oral care composition/product has increased activity to:
(i) inhibit adhesion of a bacterial cell, and/or
(ii) inhibit colonization by a bacterial cell,
wherein said increased activity is as compared to the activity of a reference oral care composition/product that does not comprise said water-soluble alpha-glucan.
